(19) United States
(12) Patent Application Publication
(10) Pub. No.: US 2005/0185799 A1
(43) Pub. Date: Aug. 25, 2005

(54) Method of monitoring equipment and alert device

(75) Inventor: Brian Bertram, Milwaukee, WI (US)

Correspondence Address: MICHAEL BEST & FRIEDRICH, LLP, 100 E WISCONSIN AVENUE, MILWAUKEE, WI, 53202, US

(73) Assignee: Breakthrough Medical Systems Inc., River Hills, WI (US)

(21) Appl. No.: 10/784,434

(22) Filed: Feb. 23, 2004

(51) Int. Cl. A61B 7/04; A61B 7/00

(52) U.S. Cl. 381/67; 381/56; 600/586

(57) ABSTRACT

A device and method that interfaces between a medical device and a call alert system. The device includes a microphone that senses an audio signal generated by the medical device when the medical device alarms.

### BACKGROUND OF THE INVENTION

An increase in the number of patients requiring medical treatment, nursing staff shortages, and poorly designed medical care facilities can contribute to inadequate patient monitoring and/or delayed treatment of a patient in medical care facilities. In some medical care facilities, alarms generated by patient monitoring and/or diagnostic devices may go unnoticed because of the remote location of the patient's room or because of inadequate sound generated by the patient monitoring and/or diagnostic devices.

Some patient monitoring and/or diagnostic devices are not capable of interfacing with a network or nurse call system. As a result, the patient alarms and device alarms generated by the patient monitoring and/or diagnostic device remain isolated to the location of the device. In addition, the patient alarms and device alarms are not transmitted to a central location where medical personnel can readily hear the alarms.

It is an unlikely task to modify equipment, such as a medical device to include the ability to interface with a network to include the capability of transmitting information to and communicating with another device.

### BRIEF DESCRIPTION OF THE INVENTION

The present invention relates to an interface between a patient monitoring and/or diagnostic device and a central alert system.

In one embodiment, the invention includes a device to alert medical personnel. The device comprises an audio sensor adapted to detect an audio signal from a medical device and an interface adapted to activate a call device in response to the detection of the audio signal.

The invention provides a method of alerting personnel that a medical device is sounding an audible tone. The method comprises detecting an audible tone generated by a medical device and activating a call device to transmit a signal to a destination.

The invention provides a device to alert medical personnel. The device comprises an audio sensor, a frequency counter, a microprocessor, and an interface. The audio sensor is adapted to detect an audio signal generated by a medical device. The frequency counter is adapted to count the number of audio signals detected by the audio sensor. The microprocessor is adapted to compare the audio signals to stored audio signals to determine the criticality of the audio signal. The interface is adapted to activate a call device after the frequency counter reaches a predetermined threshold.

The invention also provides a device to alert medical personnel. The device comprises a microphone, an adjustable filter, a frequency counter, a first relay, a second relay, and a microprocessor. The microphone is adapted to detect an audio signal generated by a medical device. The adjustable filter is adapted to pass the audio signal if the frequency of the audio signal is within a preselected range of frequencies. The frequency counter is adapted to count the number of audio signals passed by the adjustable filter. The first relay is adapted to activate a call device after the frequency counter reaches a predetermined threshold. The microprocessor is adapted to compare the audio signals to stored audio signals to determine the criticality of the audio signal. The second relay is adapted to activate the call device to transmit information related to the criticality of the audio signal.

The invention provides a system for alerting personnel. The system comprises a call device, a station in communication with the call device, and an alert device. The alert device includes an audio sensor adapted to detect an audio signal generated by a medical device, a relay, and a processor adapted to activate the relay when a predetermined number of the audio signals is detected by the audio sensor and which transmits a signal to the station to alert the personnel of the audio signal generated by the medical device.

### BRIEF DESCRIPTION OF THE DRAWINGS

FIG. 1 illustrates an intravenous pump connected to a patient in a medical care facility.

FIG. 2 is a block diagram of an interface system according to one embodiment of the invention.

FIG. 3 is a block diagram of an alert device according to one embodiment of the invention.

FIG. 4 is a flow chart illustrating the operation of the interface system of FIG. 2.

FIG. 5 is a block diagram of an alert device according to one embodiment of the invention.

FIG. 6 is a schematic diagram of an interface system according to one embodiment of the invention.

FIG. 7 is a flow chart illustrating the operation of the interface system of FIG. 6.

FIG. 8 is a flow chart illustrating the operation of the interface system of FIG. 6.

FIG. 9 is a block diagram of an interface system according to one embodiment of the invention.

FIG. 10 is a block diagram of an alert device utilized in the interface system of FIG. 9.

FIG. 11 is a block diagram of a transceiver utilized in the interface system of FIG. 9.

FIG. 12 is a flow chart illustrating the operation of the interface system of FIG. 9.

FIG. 13 is a block diagram of an interface system according to one embodiment of the invention.

FIG. 14 is a block diagram of an alert device according to one embodiment of the invention.

FIG. 15 is a block diagram of an alert device according to one embodiment of the invention.

FIG. 16 is a flow chart illustrating the operation of the interface system of FIG. 15.

FIG. 17 is block diagram of an interface system according to one embodiment of the invention.

FIG. 18 is a flow chart illustrating the operation of the interface system of FIG. 17.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limited. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings, and can include electrical connections or couplings.

In addition, it should be understood that embodiments of the invention include both hardware and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic based aspects of the invention may be implemented in software. As such, it should be noted that a plurality of hardware and software-based devices, as well as a plurality of different structural components can be utilized to implement the invention. Furthermore, and as described in subsequent paragraphs, the specific mechanical configurations illustrated in the drawings are intended to exemplify embodiments of the invention and that other alternative mechanical configurations are possible.

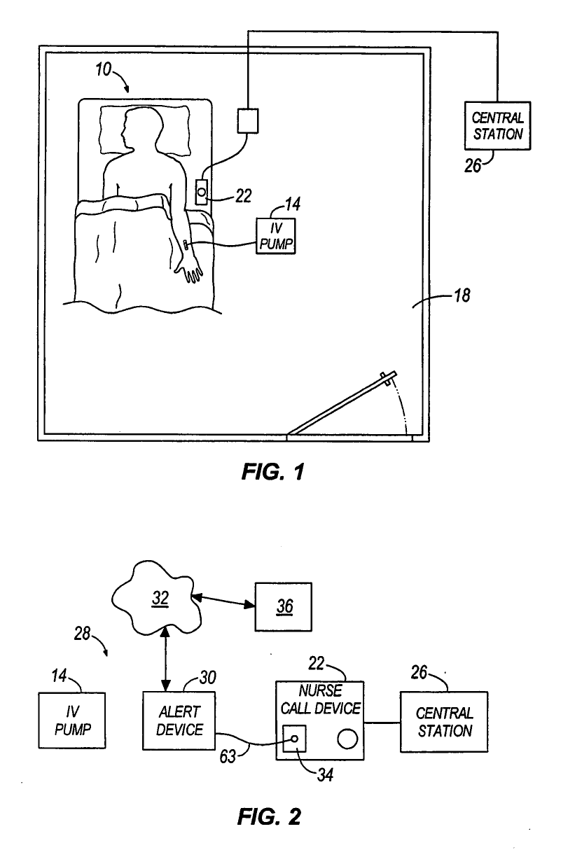

FIG. 1 illustrates a patient 10 who is connected to a patient monitoring and/or diagnostic device 14 in a medical care facility. The patient monitoring and/or diagnostic device 14 can include, but is not limited to, infusion devices, such as, an intravenous pump (“IV pump”), a PCA pump, and a pain pump; life support devices, such as, a ventilator; and vital signs monitors, such as, a patient bedside monitor, a blood pressure monitor, and an oxygen saturation monitor. The patient 10 can be connected to any number of patient monitoring and/or diagnostic devices 14.

FIG. 1 specifically illustrates the patient 10 being connected to an IV pump, which delivers fluids and medications to the patient 10. It is noted that the patient monitoring and/or diagnostic device 14 can be any device, whether or not it is specifically mentioned above. It is also noted that the embodiments of the invention are described with respect to the patient monitoring and/or diagnostic device 14 being an IV pump, however, the embodiments of the invention are not limited to use with an IV pump as described herein. The embodiments of the invention will not be described herein with respect to the patient monitoring device 14 being a different device, however, a person skilled in the art will understand how to apply the described embodiments of the invention to the other devices.

The patient 10 in FIG. 1 is located in a room 18 of a medical facility. The patient 10 can be located in any other location or building where the patient 10 can receive medical attention. Generally, each patient bed or each patient bed area, as there may be more than one bed in a room, is equipped with a central alert system, such as a nurse call device 22. The patient 10 can push a button or otherwise activate the nurse call device 22, which transmits a signal to a central station 26, to alert medical personnel that the patient 10 needs assistance. The nurse call device 22 is generally hardwired to the central station 26 behind the walls of the medical care facility. The nurse call device 22 may be hardwired to a strobe light (not shown) outside of the room 18 that illuminates upon activation of the nurse call device 22.

The nurse call device 22 includes a memory, which is generally pre-programmed with the room number of the room 18 in which the nurse call device 22 is located, and/or a bed number that it is attached to, and/or a patient number of the patient 10 in the room 18. The signal transmitted from the nurse call device 22 to the central station 26 includes the room number and/or the patient number, such that the room number, and/or the bed number, and/or the patient number can be displayed on the central station 26. The medical personnel can recognize which patient 10 has requested assistance and/or bed and/or room 18 where the assistance is needed based on the information displayed at the central station 26.

IV pumps are movable devices that can be easily transported with the patient 10. The patient 10 can remain connected to the same IV pump 14 throughout his or her stay at the medical facility. The IV pump 14 can be transported with the patient 10 when he or she needs to be transported to other areas within the medical facility for medical testing and/or medical procedures. As a result of the transportability, IV pumps generally are not designed to include the capability to connect to a network via a wireless or a wired connection.

The medical personnel can connect the patient 10 to the IV pump 14 and can make the appropriate settings, such as equipment performance parameters and patient monitoring and/or diagnostic parameters, on the IV pump 14 for the patient 10. The IV pump 14 can generate an audible tone and/or a visual indication or alarm if its operation goes outside of the set performance parameters or determines that patient data is outside of the monitoring and/or diagnostic parameters. An audible and/or a visual alarm may also be generated by the IV pump 14 if it malfunctions or for other device related problems or if supplies (i.e., IV solutions) are depleted. The visual alarm is limited to the display on the IV pump 14, and the audible alarm is limited to the confines of the room 18 in which the IV pump 14 is located. Consequently, medical personnel that are not within hearing distance of the IV pump 14 will not hear the alarm.

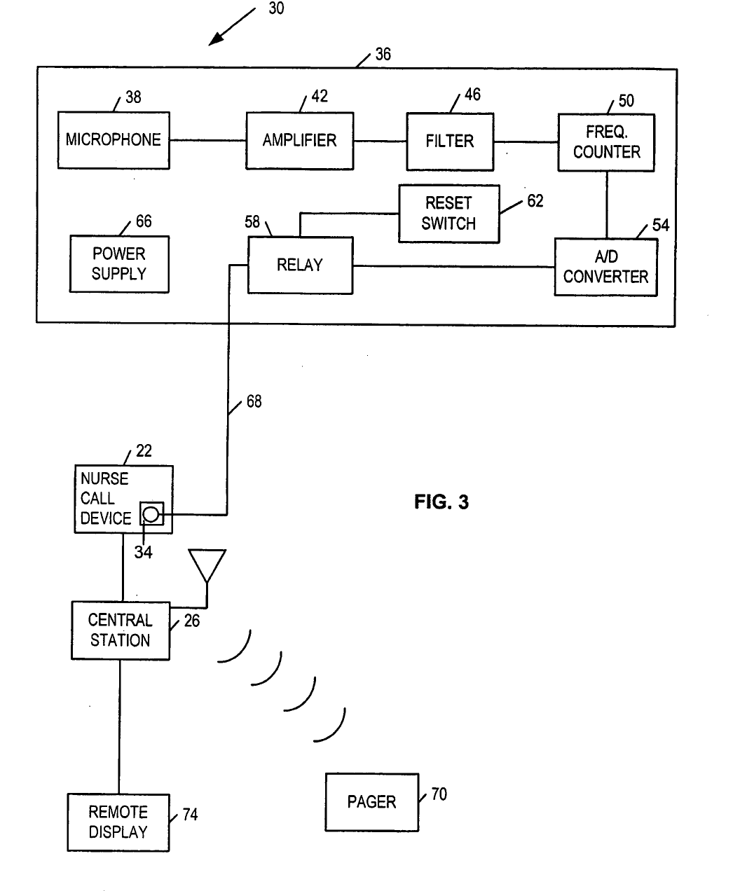

FIG. 2 illustrates a system 28, including an alert device 30 according to one embodiment of the invention that can interface between the IV pump 14 and the central station 26 to alert the medical personnel of audible tones generated by the IV pump 14. The alert device 30 can be positioned, attached, secured, or otherwise supported (e.g., with Velcro, two-sided tape, elastic rope, etc.) on or near the housing of the IV pump 14. The alert device 30 can be electrically connected to the nurse call device 22 through an auxiliary port 34 on the nurse call device 22. Most nurse call devices 22 have multiple auxiliary inputs that can be programmed to indicate what device and/or an identification of the device that is connected.

The alert device 30 can communicate with other computer systems throughout the medical facility via a network 32 as illustrated in FIG. 2. For example, when the IV pump 14 has depleted its supply of IV solutions, the IV pump 14 can communicate with a pharmacy computer system 36 to reorder IV solutions. The IV pump 14 generates an audible tone at a certain frequency or frequency range that indicates the IV solutions has been depleted. The alert device 30 can detect the audible tone and can determine (i.e., based on the frequency of the tone or through additional processing means) the meaning of the tone in order to communicate with the pharmacy computer system 36 via the network 32 and to automatically reorder the necessary supplies. Interconnectivity to a pharmacy computer system is only one example of the functionality of the alert device 30. The alert device 30 can be configured and programmed to communicate with various networks and computer systems throughout the medical care facility.

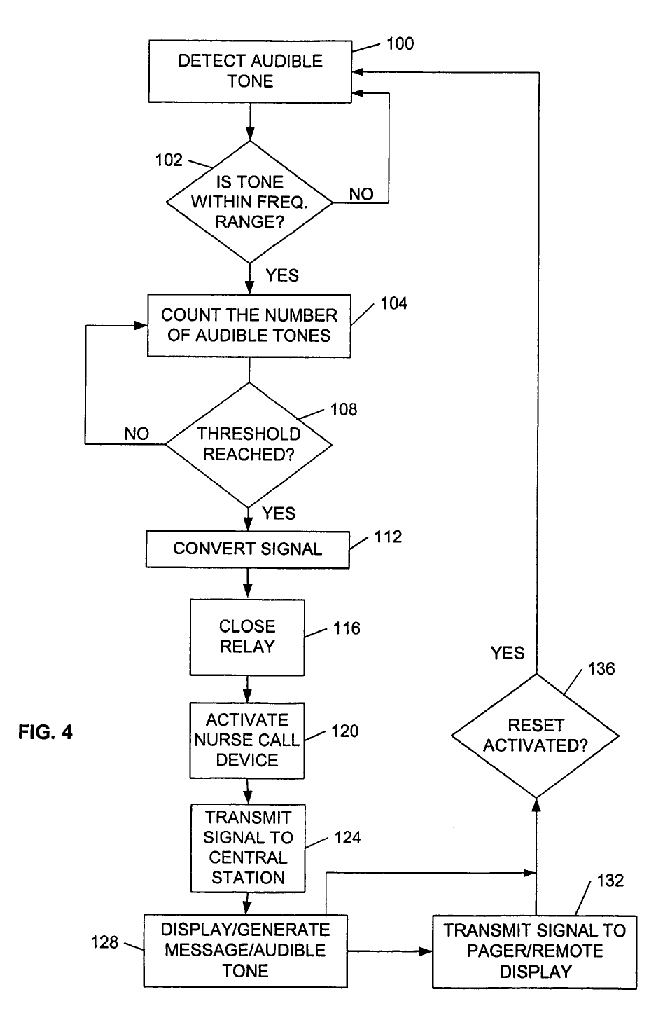

FIGS. 3-4 illustrate one embodiment of the alert device 30. Generally, the alert device 30 includes a housing 36 and a microphone 38 (or microphones that are positioned internally and/or externally of the device 30) adapted to sense and/or detect sound from an audible tone generated by the IV pump 14. The alert device 30 includes an amplifier 42 electrically connectable to the microphone 38. The amplifier 42 is operable to amplify the signal sensed and/or detected by the microphone 38. The alert device 30 includes a filter 46 electrically connectable to the amplifier 42. The filter 46 is operable to allow certain signal frequencies of the audible tone to pass. The alert device 30 includes a frequency counter 50 electrically connectable to the filter 46. The frequency counter 50 is operable to count the number of alarm tones sensed and/or detected by the microphone 38 that have been transmitted through the filter 46. The alert device 30 includes an analog to digital converter 54 to convert the analog signal to a digital signal. The alert device 30 includes a relay 58 (e.g., a DC solid-state relay). The relay 58 is in a normally open position and is operable to close and latch, which activates the nurse call device 22. The alert device 30 includes a reset switch 62 electrically connectable to the relay 58. The reset switch 62 is operable to reset the relay 58 to the normally open position. Alternatively, the alert device 30 can include a window (not shown) to receive an infrared signal from a remote reset switch (e.g., a keyfob-like device) to reset the relay 58 to the normally open position. The alert device 30 includes a power supply 66. The alert device 30 includes an electronic cord 68 adapted to be electrically connected to the auxiliary port 34 on the nurse call device 22. The alert device 30 is generally positioned near a speaker on the IV pump 14.

With reference to FIG. 4, the microphone 38 of the alert device 30 can sense and/or detect (at step 100) an audible tone generated by the IV pump 14. The filter 46 determines (at 102) whether the frequency of the audible tone is within the frequency range of the filter. If the frequency of the audible tone is not within the frequency range of the filter 46, the alert device 30 ignores the audible tone. If the frequency is within the frequency of the filter 46, the frequency counter 50 counts (at 104) the number of audible tones detected by the microphone 38 and transmitted through the filter 46. If the number of audible tones reaches (at 108) a certain threshold, the analog signals are converted (at 112) to digital signals by the analog to digital converter 54. The signals cause the relay 58 to close (at 116), which activates (at 120) the nurse call device 22 to send a signal to the central station 26 and/or illuminate the light outside of the room 18. The signal that is transmitted (at 124) from the nurse call device 22 to the central station 26 can include the room number, and/or the bed number, and/or the patient number that is stored in memory of the nurse call device 22. The central station 26 displays and/or generates (at 128) a message and/or an audible tone that includes the room number in which the IV pump 14 is located, and/or bed number that the IV pump 14 is assigned to, and/or patient number that the IV pump 14 is assigned to, to alert the medical personnel that the IV pump 14 is in an alarm state.

In addition and as shown in FIGS. 3 and 4, the central station 26 generates (at 132) a signal that can be transmitted to a pager 70 and/or remote display 74 to alert remote medical personnel of the need for assistance in room 18. The pager 70 and/or remote display 74 can display the room number, and/or the bed number, and/or the patient number to inform the medical personnel of the location where his or her assistance is needed. The medical personnel can activate (at 136) the reset switch 62 on the alert device 30 to reset the alert device 30 and stop the transmission of the signal to the central station 26.

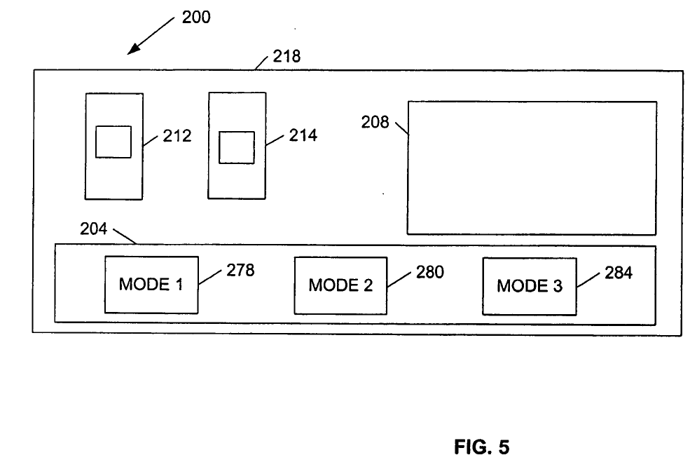

FIGS. 5-8 illustrate another embodiment of the invention as alert device 200. The alert device 200 includes a keypad 204 for the selection of various modes of operation as will be discussed below. The alert device 200 includes a display 208 (e.g., a LCD display) to identify the mode of operation selected. The display 208 can display additional information, such as location (e.g., the room number), equipment identification number, an IV pump identification number which the alert device 200 is connected, patient identification number, patient information, number and/or type of alarm tones detected, history of operation, times of operation, data from the other computer system, and alarm data, etc.

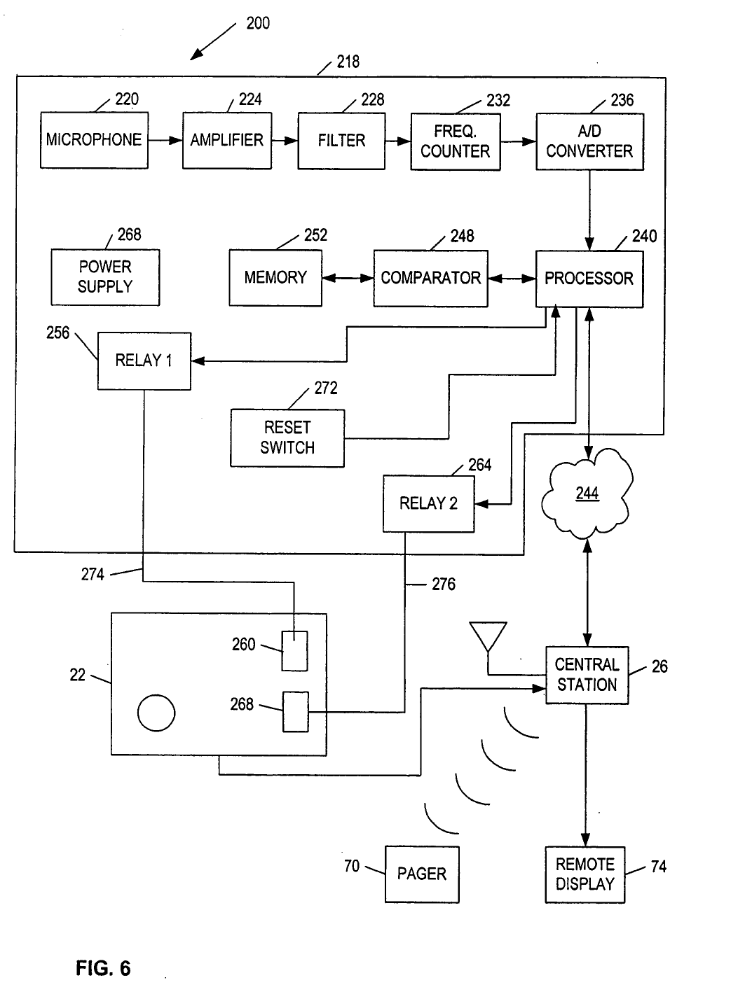

The alert device 200 includes a slide switch 212, button, or other like device electrically connectable to an adjustable filter (illustrated in FIG. 6). A user and/or medical personnel can adjust the switch 212 to select the range of frequencies of the audible tones generated by the IV pump 14 that are desirable for detection and notification at the central station 26. The alert device 200 includes a time delay switch 214, button, or other like device electrically connectable to a microprocessor (illustrated in FIG. 6). A user and/or medical personnel can adjust the switch 214 to select the amount of time delay before the central station 26 receives a signal indicating that the IV pump 14 is in an alarm state.

As illustrated in FIG. 6, the alert device 200 includes a housing 218 and a microphone 220 adapted to sense or detect sound from an audible tone generated by the IV pump 14. The alert device 200 includes an amplifier 224 electrically connectable to the microphone 220. The amplifier 224 is operable to amplify the signal detected by the microphone 220. The alert device 200 includes a filter 228 (e.g., adjustable filter) electrically connectable to the amplifier 224. The filter 228 is operable to allow certain signal frequencies of the audible tone to pass. The frequency or range of frequencies that the filter 228 passes can be selected by the slide switch 212. The alert device 200 includes a frequency counter 232 electrically connectable to the filter 228. The frequency counter 232 is operable to count the number of audible tones sensed and/or detected by the microphone 220 and that have been transmitted through the filter 228. The alert device 200 includes an analog to digital converter 236 to convert the analog signal to a digital signal.

The alert device 200 includes a microprocessor 240 electrically connectable to the analog to digital converter 236. The microprocessor 240 can be connectable to a network 244, such as an Ethernet network for programming the microprocessor 240. The network 244 may be the same as or different than the network 32 discussed above. The microprocessor 240 can be programmed by the central station 26 and/or a separate computer terminal capable of communicating with the microprocessor 240. The alert device 200 includes a comparator 248 electrically connectable to the microprocessor 240. The comparator 248 compares the detected alarm tone(s) to a set of stored and/or sampled alarm tones in a memory 252, such as an EEPROM or external memory card (e.g., a smart media memory card). This external memory card or EEPROM could also be pre-recorded with known tones of devices from a library of devices and tones. In addition, the alert device 200 can be configured to include a PCMCIA slot for insertion of an ethernet, USB, or Firewire card. The alert device 200 can use the ethernet, USB, or Firewire card to periodically connect to a specific computer via the network 244 or to the memory card via a smart media reader to download and update the known audible tones from a web site or web sites.

The alert device 200 includes a first relay 256 (e.g., a DC solid-state relay). The first relay 256 is in a normally open position and is operable to close and latch, which activates the nurse call device 22. The first relay 256 is electrically connectable to the nurse call device 22 via a first auxiliary port 260 on the nurse call device 22. The alert device 200 includes a second relay 264 (e.g., a DC solid-state relay). The second relay 264 is in a normally open position and is operable to close and latch, which also activates the nurse call device 22. The second relay 264 is electrically connectable to the nurse call device 22 via a second auxiliary port 268 on the nurse call device 22.

The alert device 200 includes a reset switch 272 electrically connectable to the microprocessor 240. The reset switch 272 is operable to direct the microprocessor 240 to reset the first relay 256 and/or the second relay 264 to the normally open position. Alternatively, the alert device 200 can include a window (not shown) to receive an infrared or RF signal from a remote reset switch (e.g., a keyfob-like device) to reset the first relay 256 and/or the second relay 264 to the normally open position. The alert device 200 includes a power supply 268 to provide power to the microprocessor 240. The power supply 268 can be electrically coupled to a power source, such as a 110 VAC power outlet and/or a battery. The alert device 200 includes an electronic cord 274 adapted to be electrically connected between the first relay 256 and the auxiliary port 260 on the nurse call device 22. The alert device 200 includes a second electronic cord 276 adapted to be electrically connected between the second relay 264 and the auxiliary port 268.

The alert device 200 can include multiple operational modes. To accommodate the multiple operational modes, the alert device 200 includes multiple keypad selections 278, 280, and 284 on the keypad 204. The embodiment of the alert device 200 shown in FIG. 5 illustrates three keypad selections; however, the alert device 200 can include fewer or more keypads and modes of operation than illustrated.

An operator can select the keypad 278 for a first mode of operation of the alert device 200. The first mode of operation (e.g., auto mode or automatic mode) has been generally described above and as illustrated in FIG. 4 for the alert device 30. The microphone 220 senses and/or detects (at 100) an audible tone generated by the IV pump 14. The filter 46 determines (at 102) whether the frequency of the audible tone is within the frequency range of the filter. If the frequency of the audible tone is not within the frequency range of the filter 46, the alert device 30 ignores the audible tone. If the frequency is within the frequency of the filter 46, the frequency counter 50 counts (at 104) the number of audible tones detected by the microphone 38 and transmitted through the filter 46. If the number of audible tones reaches (at 108) a certain threshold, the analog signals are converted (at 112) to digital signals by the analog to digital converter 54. The signals cause the first relay 256 to close (at 116), which activates (at 120) the nurse call device 22. The signal that is transmitted (at 124) from the nurse call device 22 to the central station 26 can include the room number, and/or the bed number, and/or the patient number. The central station 26 displays and/or generates (at 128) a message and/or an audible tone that includes the room number in which the IV pump 14 is located, and/or bed number that the IV pump 14 is assigned to, and/or patient number that the IV pump 14 is assigned to, to alert the medical personnel that the IV pump 14 is in an alarm state. In addition, the central station 26 can generate (at 132) a signal that can be transmitted to a pager 70 and/or remote display 74 to alert remote medical personnel of the need for assistance in room 18. The pager 70 and/or remote display 74 can display the room number, and/or the bed number, and/or the patient number to inform the medical personnel of the location where his or her assistance is needed. The medical personnel can activate (at 136) the reset switch 272 on the alert device 200 to reset the first relay 256 and the alert device 200 and to stop the transmission of the signal to the central station 26 (and the pager 70 and/or the remote display 74).

An operator can select the keypad 280 for a second mode of operation of the alert device 200. The second mode of operation (e.g., sample mode) can utilize sound sampling technology to detect different types of audible tones (i.e., a different frequency or different frequency ranges). The microprocessor 240 samples and/or records multiple audible tones of the IV pump 14 (or any other device) and stores the sampled audible tones in the memory 252. The microprocessor 240 and/or memory 252 can store the audible tones based on frequency and/or duration and/or other detectable attributes of the audible tones. The memory 252 can be memory within the microprocessor 240 or a separate memory component or device. The memory 252 can also be a component or device that is external to the alert device 200. The microprocessor 240 is programmed to identify the stored audible tones by type and/or category (i.e., the criticality of the alarm tones, such as low, medium, and high or any other desirable naming convention). For example, a bedside patient monitor can generate different audible tones based on the nature of the problem associated with the audible tone. The monitor can generate a first audible tone at one frequency and duration for asystole and a second audible tone at a different frequency and duration for bradycardia. The audible tone for asystole would generally represent a critical alarm, whereas the audible tone for bradycardia would generally represent a less critical alarm than the asystole alarm.

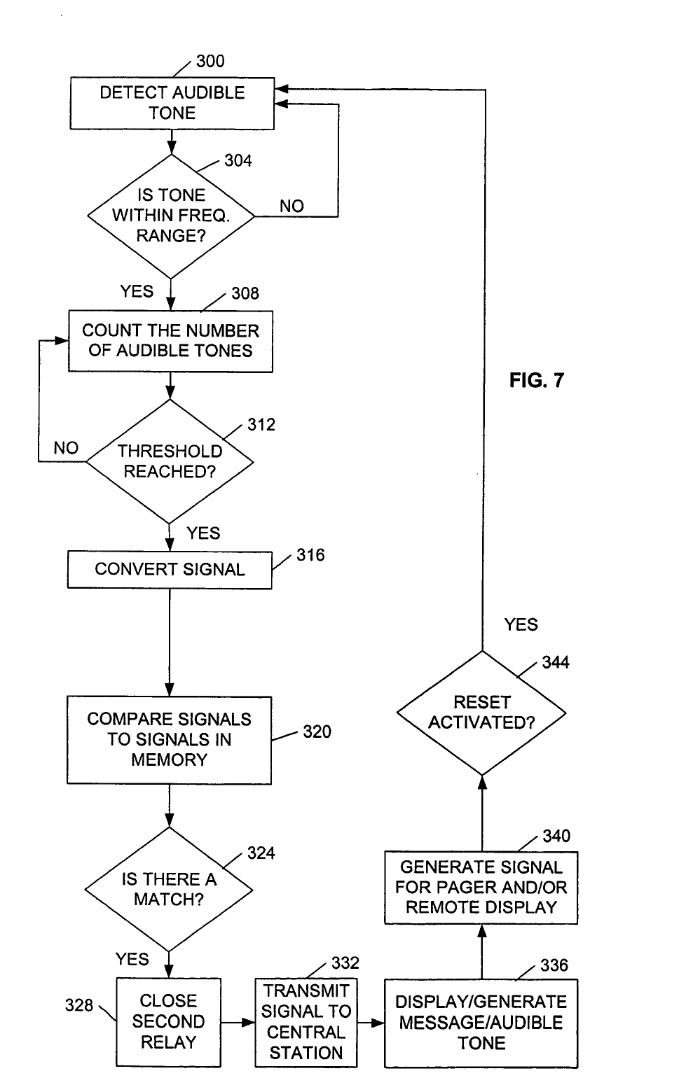

Referring to FIG. 7, the microphone 220 of the alert device 200 senses and/or detects (at step 300) an audible tone generated by the IV pump 14. The adjustable filter 228 is preset by the medical personnel based on the position of the switch 212. The adjustable filter 228 determines (at 304) if the frequency of the audible tone is within the frequency range set by the switch 212. If the frequency of the audible tone is not within the frequency range set by the switch 212, the alert device 200 ignores the audible tone. If the frequency is within the frequency range set by the switch 212, the frequency counter 232 counts (at 308) the number of audible tones detected by the microphone 220 and transmitted through the adjustable filter 216. If the frequency counter 232 reaches (at 312) a certain threshold, the analog signals are converted (at 316) to digital signals by the analog to digital converter 236. The microprocessor 240 and the comparator 248 receive the digital signals such that the comparator 248 compares (at 320) the signals to the signals stored in the memory 252 to determine the criticality of the alarm tone. If the digital signals match (at 324) any of the signals stored in the memory 252, the microprocessor 240 transmits a signal, which includes the criticality of the audible tone, to cause the second relay 264 to close (at 328). The signal activates the nurse call device 22 to transmit (at 332) a signal to the central station 26 and/or illuminate the light outside of the room 18. Alternatively, the microprocessor 240 can transmit a signal, which includes the criticality of the audible tone, to the central station 26 via the network 244.

The central station 26 receives the signal including the criticality of the audible tone to alert the medical personnel that the IV pump 14 (or other device, such as a bedside or patient monitor) is in an alarm state and to inform the medical personnel of the severity of the alarm condition. The central station 26 can also receive the room number, and/or the bed number, and/or the patient number. The central station 26 displays and/or generates (at 336) a message and/or an audible tone to alert the medical personnel of the room number in which the IV pump 14 is located, and/or bed number that the IV pump 14 is assigned to, and/or patient number that the IV pump 14 is assigned to. The central station 26 generates a message (e.g., brighter, bolder, flashing, etc.) and/or audible tone (e.g., louder, different tone, faster beeping, etc.) to represent the criticality of the audible tone generated by the IV pump 14. In addition, the central station 26 can generate (at 340) a signal that can be transmitted to a pager 70 and/or remote display 74 to alert remote medical personnel of the critical nature of the assistance needed in room 18. The pager 70 and/or remote display 74 can display the room number, and/or the bed number, and/or the patient number with the identification of the critical nature of the audible tone (e.g., brighter, bolder, flashing message and/or louder, different tone, faster beeping, audible tone) to inform the medical personnel of the location where his or her assistance is needed. The medical personnel can activate (at 344) the reset switch 272 on the alert device 200 to reset the second relay 264 and the alert device 200 and stop the transmission of the signal to the central station 26.

An operator can select keypad 284 for a third mode of operation of the alert device 200. The third mode of operation (e.g., dual mode) generally includes the operational features of the first mode of operation and the second mode of operation that have been described above for the alert device 200.

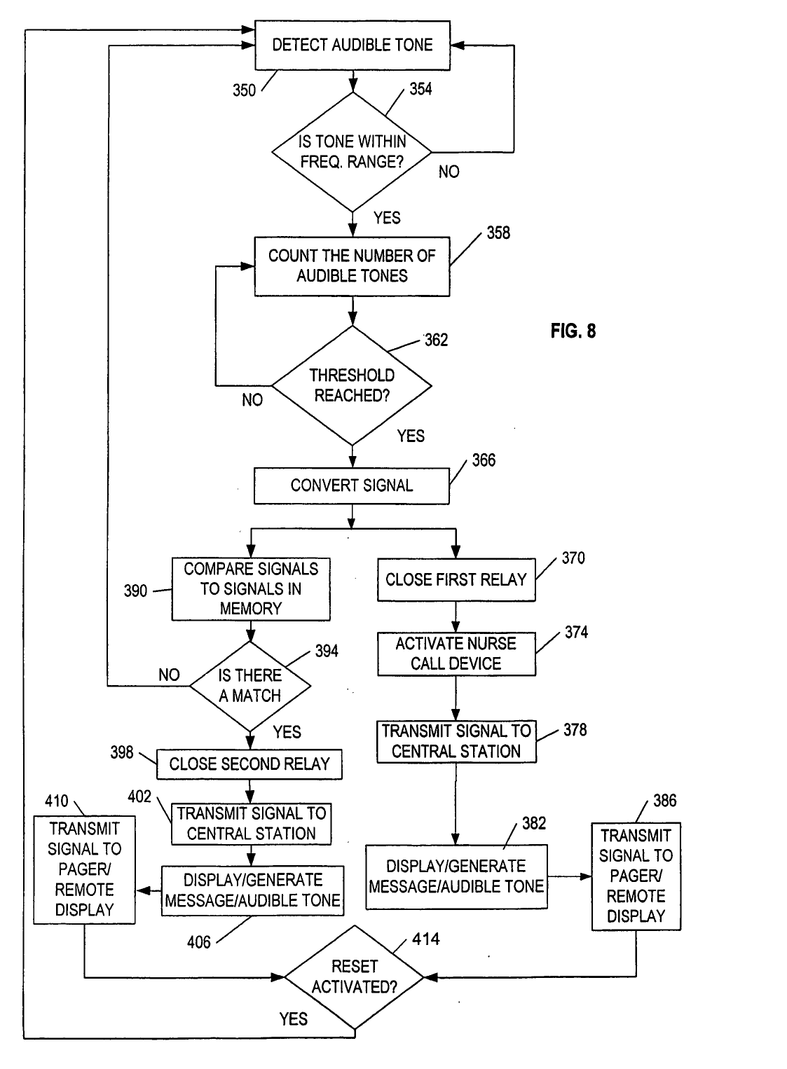

Referring to FIG. 8, which illustrates the operation of the alert device 200 in the third mode, the microphone 220 of the alert device 200 senses and/or detects (at 350) an audible tone generated by the IV pump 14. The adjustable filter 228 is preset by the medical personnel based on the position of the switch 212. The adjustable filter 228 determines (at 354) if the frequency of the audible tone is within the frequency range set by the switch 212. If the frequency of the audible tone is not within the frequency range set by the switch 212, the alert device 200 ignores the audible tone. If the frequency of the audible tone is within the frequency range set by the switch 212, the frequency counter 232 counts (at 358) the number of audible tones detected by the microphone 220 and transmitted through the adjustable filter 228. If the frequency counter 232 reaches (at 362) a certain threshold, the analog signals are converted (at 366) to digital signals by the analog to digital converter 236. The microprocessor 240 receives the digital signals, which causes the first relay to close (at 370), which activates (at 374) the nurse call device 22. The signal that is transmitted (at 378) from the nurse call device 22 to the central station 26 can include the room number, and/or the bed number, and/or the patient number that is stored in the nurse call device 22. The central station 26 displays and/or generates (at 382) a message and/or an audible tone that includes the room number in which the IV pump 14 is located, and/or bed number that the IV pump 14 is assigned to, and/or patient number that the IV pump 14 is assigned to, to alert the medical personnel that the IV pump 14 is in an alarm state. In addition, the central station 26 generates (at 386) a signal that can be transmitted to a pager 70 and/or remote display 74 to alert remote medical personnel of the need for assistance in room 18. The pager 70 and/or remote display 74 can display the room number, and/or the bed number, and/or the patient number to inform the medical personnel of the location where his or her assistance is needed.

After the signals are converted to digital signals (at 366), the comparator 248 also receives the digital signals and compares (at 390) the signals to the signals stored in the memory 252 to determine the criticality of the alarm tone. If the digital signals match (at 394) any of the signals stored in the memory 252, the microprocessor 240 transmits a signal, which includes the criticality of the audible tone, to cause the second relay 264 to close (at 398). The signal activates the nurse call device 22 to transmit (at 402) a signal to the central station 26. The central station 26 receives the signal including the criticality of the audible tone to alert the medical personnel that the IV pump 14 is in an alarm state and informs the medical personnel of the severity of the alarm condition. The central station 26 can also receive the room number, and/or the bed number, and/or the patient number that is transmitted with the signal. The central station 26 displays and/or generates (at 406) a message and/or an audible tone to alert the medical personnel of the room number in which the IV pump 14 is located, and/or bed number that the IV pump 14 is assigned to, and/or patient number that the IV pump 14 is assigned to. The central station 26 generates a separate message (e.g., brighter, bolder, flashing, etc.) and/or audible tone (e.g., louder, different tone, faster beeping, etc.) to represent the criticality of the alarm tone. Alternatively, the central station 26 can overwrite the message and/or audible tone from the previous alarm message and/or audible tone to indicate the criticality of the alarm tone. In addition, the central station 26 can generate (at 410) a signal that can be transmitted to a pager 70 and/or remote display 74 to alert remote medical personnel of the criticality of the need for assistance in room 18. The pager 70 and/or remote display 74 can display the room number, and/or the bed number, and/or the patient number to inform the medical personnel of the location where his or her assistance is needed. The medical personnel can activate (at 414) the reset switch 272 on the alert device 200 to reset the alert device 200 and stop the transmission of the signal to the central station 26.

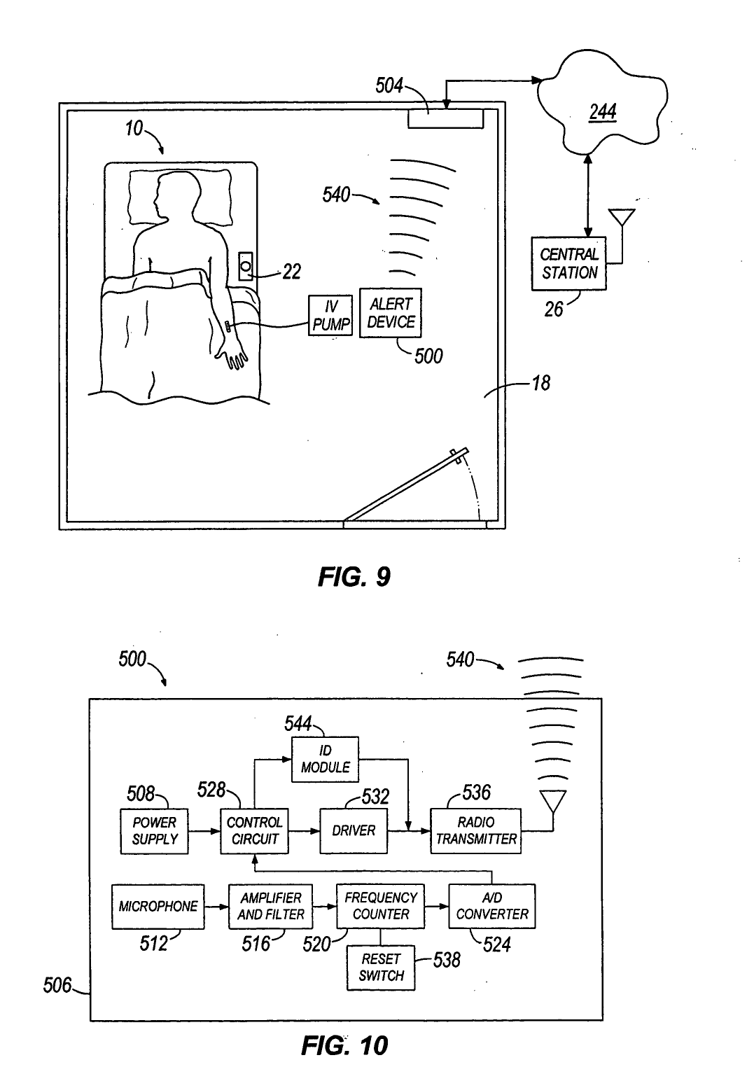

FIGS. 9-12 illustrate another embodiment of the invention as alert device 500. The alert device 500 is configured for wireless communication with the central station 26. The room 18 can include one or more antennas and/or transceivers 504 to transmit communications between the alert device 500 and the central station 26. The wireless communication between the alert device 500 and the central station 26 can occur using infrared, ultrasonic, radio frequency, or any other method of wireless communication between two devices.

Referring to FIG. 10, the alert device 500 includes a housing 506 and a power supply 508, which can be electrically coupled to a power source, such as a 110 VAC power outlet and/or a battery. The alert device 500 includes a microphone 512 adapted to sense and/or detect sound from an audible tone generated by the IV pump 14. The alert device 500 includes an amplifier and filter circuit 516 electrically connectable to the microphone 512. The amplifier and filter circuit 516 are operable to amplify the signal sensed and/or detected by the microphone 512 and to filter the signal to allow certain signal frequencies of the audible tone to pass. The alert device 500 includes a frequency counter 520 electrically connectable to the amplifier and filter circuit 516. The frequency counter 520 is operable to count the number of audible tones sensed and/or detected by the microphone 512 that have been transmitted through the amplifier and filter circuit 516. The alert device 500 includes an analog to digital converter 524 electrically connected to the frequency counter 520. The analog to digital converter 524 is operable to convert analog signals to digital signals. The alert device 500 includes a control circuit 528 electrically connected to the power supply 508 and the A/D converter 524. The control circuit 528 can be a microprocessor, other programmable device, or an application specific integrated circuit (“ASIC”). The alert device 500 includes a driver 532 electrically connected to the control circuit 528. The driver 532 can be a tone generator or a pulse generator or other type of generator based on the type of communication between the alert device 500 and the transceiver 504.

The alert device 500 includes a radio transceiver 536 electrically connected to the driver 532. The radio transceiver 536 can be a transducer, infrared device, or other radiation-emitting device. The driver 532 causes the radio transceiver 536 to transmit or broadcast a signal 540 into the room 18. The alert device 500 includes an identification module 544 electrically connected to the control circuit 528. The identification module 544 can include a unique code that can generally represent the room number where the alert device 500 is located, the patient number that is assigned to the alert device 500, and/or the equipment identification number that is assigned to the alert device 500. The identification module 544 can be programmed or preset to include any identification means desired by the user and/or medical personnel. The control circuit 528 controls the identification module 544 to include, attach, and/or combine the unique code with the signal 540, which can then be transmitted to the transceiver 504. Alternatively, the unique code can be programmed into the control circuit 528 such that the unique code is automatically included, attached, and/or combined with the signal 540. The radio transceiver 536 can receive communication signals from the transceiver 504 and allow two-way communication between the transceiver 504 and the alert device 500. The central station 26 includes a reset switch 538 (as illustrated in FIG. 9) that is operable to reset the alert device 500. Medical personnel can activate the reset switch 538 at the central station 26, which generates a signal that is transmitted to the radio transceiver 536 via the network 244 and transceiver 504. The radio transceiver 536 transmits the signal to the control circuit 528 to reset the alert device 500. Alternatively, the reset switch 538 can be electrically connectable to the control circuit 528 that is operable to reset the alert device 500.

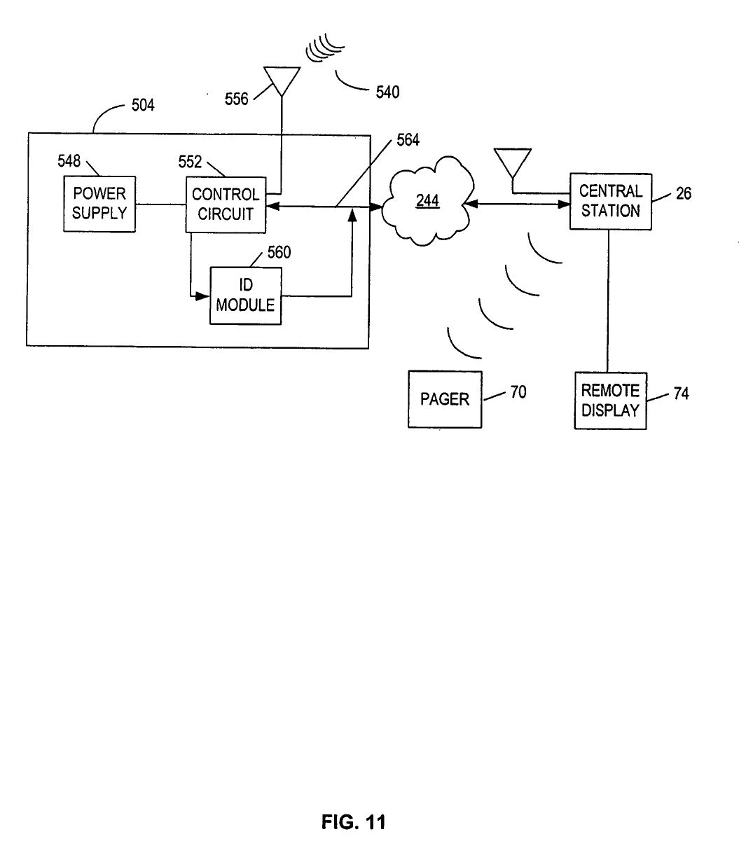

Referring to FIG. 11, the transceiver 504 includes a power supply 548, which can be electrically coupled to a power source (not shown), such as a 110 VAC power outlet and/or a battery. The transceiver 504 includes a control circuit 552 electrically connected to the power supply 548. The control circuit 552 can be a microprocessor, other programmable device, or an ASIC. The transceiver 504 includes an amplifier and filter circuit (not shown). The amplifier and filter circuit are operable to amplify and filter the incoming signal 540 from the alert device 500 prior to re-transmission to the central station 26. The transceiver 504 includes an antenna 556. The antenna 556 is operable to sense and/or detect incoming signals 540 from the alert device 500. The transceiver 504 includes an identification module 560. The control circuit 552 controls the identification module 560 to include and/or combine a unique code with the signal, which can be transmitted via the network 244 to the central station 26. Alternatively, the unique code can be programmed into the control circuit 552 via the network 244, and the unique code can generally represent the room number where the alert device 500 is located, the patient number that is assigned to the alert device 500, and/or the equipment identification number that is assigned to the alert device 500. The alert device 500 is generally positioned near a speaker on the IV pump 14.

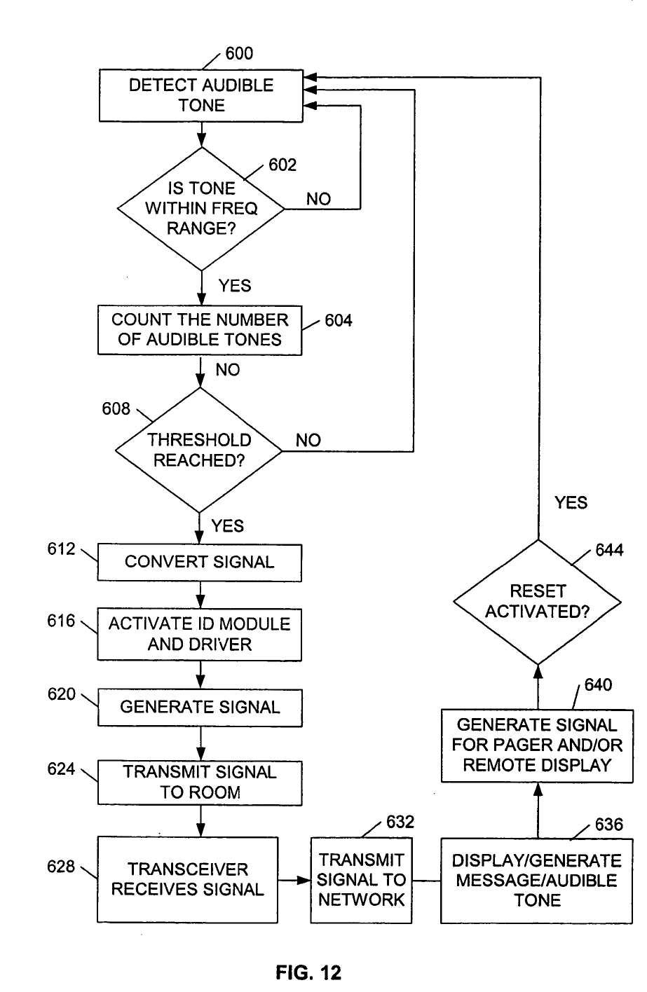

With reference to FIG. 12, the microphone 512 of the alert device 500 senses and/or detects (at step 600) an audible tone generated by the IV pump 14. The amplifier and filter circuit 516 determines (at 602) whether the frequency of the audible tone is within the frequency range of the filter within the amplifier and filter circuit 516. If the frequency of the audible tone is not within the frequency range of the filter, the alert device 500 ignores the audible tone. If the frequency is within the frequency of the filter, the frequency counter 520 counts (at 604) the number of audible tones detected by the microphone 512 and transmitted through the amplifier and filter circuit 516. If the frequency counter 520 reaches (at 608) a certain threshold, the analog signals are converted (at 612) to digital signals by the analog to digital converter 524. The control circuit 528 receives the digital signals and activates (at 616) the ID module 544 and the driver 532. The ID module 544 combines the unique code (stored in memory) with the signal output from the driver 532 to generate (at 620) the signal 540. The radio transceiver 536 receives the signal 540 and transmits (at 624) the signal 540 with the unique code into the room 18. The control circuit 552 of the transceiver 504 (illustrated in FIG. 11) receives (at 628) the signal 540. The signal 540 can be further processed by the transceiver 504 as necessary.

The control circuit 552 can activate the ID module 560 to combine a unique code (the same or different code than the unique code from the alert device 500) with the signal 540 (the ID module 560 can combine the unique code with the signal 540 whether or not the ID module 544 of the alert device 500 has combined the unique code to the signal 540) to generate a signal 564. The signal 564 is transmitted (at 632) from the transceiver 504 to the central station 26 via the network 244. The central station 26 displays and/or generates (at 636) a message and/or an audible tone that includes the unique code, which can represent the location of the IV pump 14, and/or bed number that the IV pump 14 is assigned to, and/or the patient number that the IV pump 14 is assigned to, to alert the medical personnel that the IV pump 14 is in an alarm state. In addition, the central station 26 can generate (at 640) a signal that can be transmitted to a pager 70 and/or remote display 74 to alert remote medical personnel of the need for assistance in room 18. The pager 70 and/or remote display 74 can display the room number, and/or the bed number, and/or the patient number to inform the medical personnel of the location where his or her assistance is needed. The medical personnel can activate (at 644) the reset switch 538 on the alert device 500 to reset the alert device 500 and stop the transmission of the signal to the central station 26.

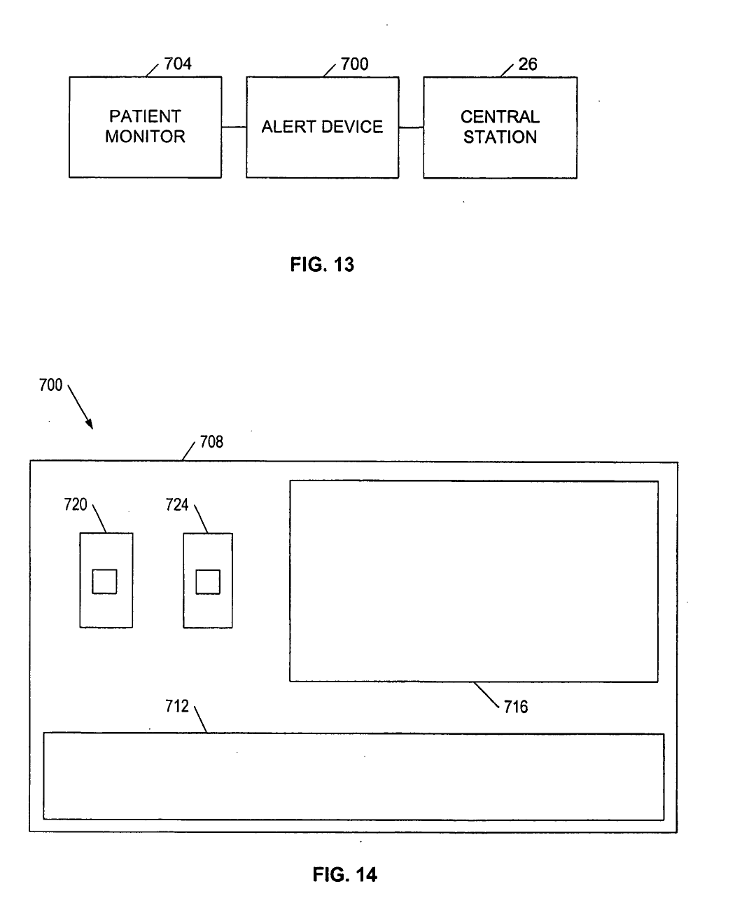

FIGS. 13-16 illustrate another embodiment of the invention as alert device 700. The alert device 700 is electrically connected to a patient monitor 704 (i.e., vital signs patient monitor, blood pressure monitor, pulse oximetry monitor, etc.) and the central station 26. The alert device 700 can be electrically connected to the patient monitor 704 and the central station 26 via a serial communications port, such as an RS232 port. Alternatively, the alert device 700 can include an infrared port to receive infrared signals or a transceiver to receive RF signals from the patient monitor 704. The alert device 700 would include the necessary electronic systems and/or components embodied in hardware and/or software to accommodate the infrared or RF communication modes.

Referring to FIG. 14, in one embodiment, the alert device 700 includes a housing 708. The alert device 700 includes a keypad 712 for the selection of various modes of operation, and to provide input data and/or input instructions for operation of the alert device 700, as will be discussed below. The alert device 700 includes a display 716 (e.g., a LCD display) to identify the mode of operation selected. The display 716 can display additional information, such as location (e.g., the room number), equipment identification number, a patient monitor identification number which the alert device 700 is connected, patient identification number, patient information, number and/or type of alarm tones detected, history of operation, times of operation, data from the other computer system, alarm data, questions to the user, etc.

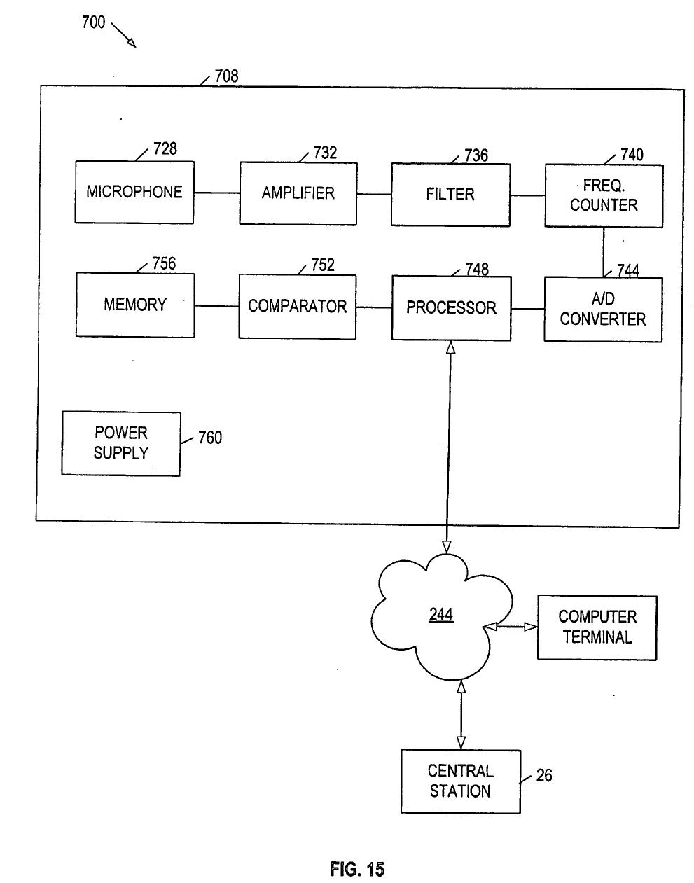

The alert device 700 includes a slide switch 720, button, or other like device electrically connectable to an adjustable filter (illustrated in FIG. 15). A user and/or medical personnel can adjust the switch 720 to select the range of frequencies of the audible tones generated by the patient monitor 704 that are desirable for detection and notification at the central station 26. The alert device 700 includes a time delay switch 724, button, or other like device electrically connectable to a microprocessor (illustrated in FIG. 15). A user and/or medical personnel can adjust the switch 724 to select the amount of time delay before the central station 26 receives a signal indicating that the patient monitor 704 is in an alarm state.

Referring to FIG. 15, in one embodiment, the alert device 700 includes a microphone 728 adapted to sense or detect sound from an audible tone generated by the patient monitor 704. The alert device 700 includes an amplifier 732 electrically connectable to the microphone 728. The amplifier 732 is operable to amplify the signal detected by the microphone 728. The alert device 700 includes a filter 736 (e.g., adjustable filter) electrically connectable to the amplifier 732. The filter 736 is operable to allow certain signal frequencies of the audible tone to pass. The frequency or range of frequencies that the filter 736 passes can be selected by the slide switch 720. The alert device 700 includes a frequency counter 740 electrically connectable to the filter 736. The frequency counter 740 is operable to count the number of audible tones sensed and/or detected by the microphone 728 and that have been transmitted through the filter 736. The alert device 700 includes an analog to digital converter 744 to convert the analog signal to a digital signal.

The alert device 700 includes a microprocessor 748 (i.e., PIC processor or a plurality of processors) electrically connectable to the analog to digital converter 744. The microprocessor 748 can be connectable to a network 244, such as an Ethernet network for programming the microprocessor 748 and transmitting data to other devices connected to the network 244. The network 244 may be the same as or different than the network 32 discussed above. The microprocessor 748 can be programmed by the central station 26 and/or a separate computer terminal capable of communicating with the microprocessor 748. Each time the alert device 700 is connected to a patient monitor 704, the processor 748 detects the new connection and requests input from the user, such as patient identification, bed identification, patient monitor identification, room identification, and other data as desired.

The alert device 700 includes a comparator 752 electrically connectable to the microprocessor 748. The comparator 752 compares the detected alarm tone(s) to a set of stored and/or sampled alarm tones in a memory 756, such as an EEPROM or external memory card. The external memory card or EEPROM could also be pre-recorded with known tones of devices from a library of devices and tones.

The alert device 700 includes a power supply 760 to provide power to the microprocessor 748. The power supply 760 can be electrically coupled to a power source, such as a 110 VAC power outlet and/or a battery. The alert device 700 can be electrically connected to a battery back-up system (not shown).

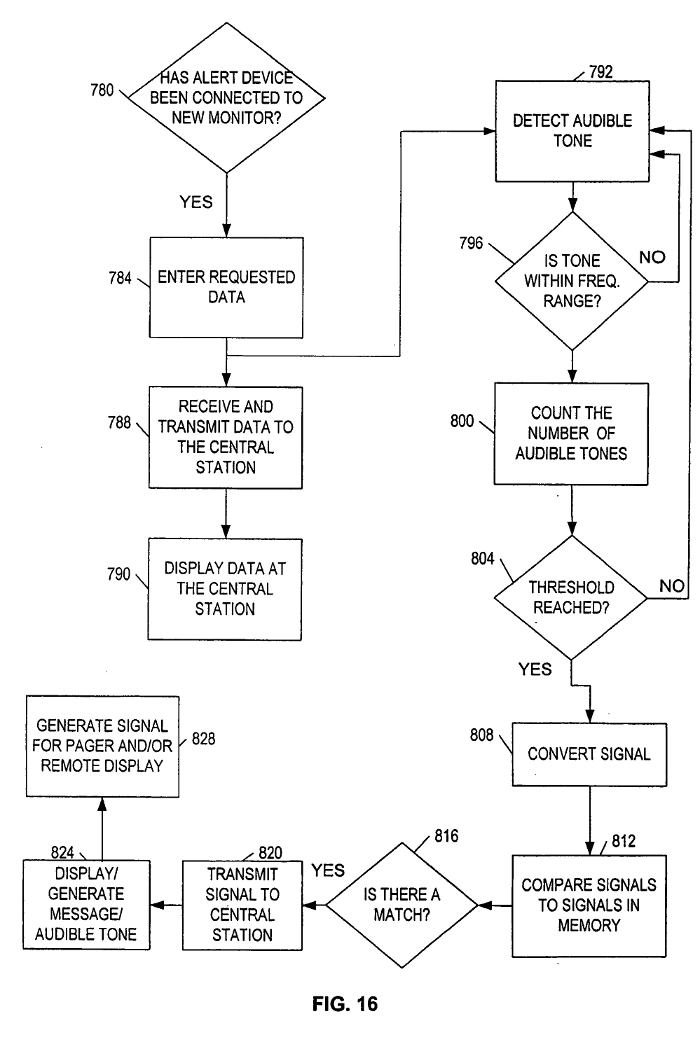

Referring to FIG. 16, the alert device is connected (or it remains wireless is utilizing infrared or RF communication technology) to a patient monitor 704. The processor 748 determines (at 780) that the alert device 700 has been connected to a new/different patient monitor 704 and requests (at 784) input data, such as patient identification, bed identification, patient monitor identification, room identification and/or other data, from the user. The requests are presented on the display 716 and the user utilizes the keypad 712 to input the requested data. After the requested data has been entered, the processor 748 receives (at 788) data (e.g., ECG, blood pressure, pulse oximetry, etc.) from the patient monitor 704 and transmits the data to the central station 26, either directly or via the network 244. The central station 26 can display (at 790) the data from the patient monitor 704. While the processor 148 is receiving data and transmitting data to the central station 26, the microphone 728 of the alert device 700 senses and/or detects (at 792) an audible tone generated by the patient monitor 704. The adjustable filter 736 is preset by the medical personnel based on the position of the switch 720. The adjustable filter 736 determines (at 796) if the frequency of the audible tone is within the frequency range set by the switch 720. If the frequency of the audible tone is not within the frequency range set by the switch 720, the alert device 700 ignores the audible tone. If the frequency of the audible tone is within the frequency range set by the switch 720, the frequency counter 740 counts (at 800) the number of audible tones detected by the microphone 728 and transmitted through the adjustable filter 736. If the frequency counter 740 reaches (at 804) a certain threshold, the analog signals are converted (at 808) to digital signals by the analog to digital converter 744. The microprocessor 748 and the comparator 752 receive the digital signals such that the comparator 752 compares (at 812) the signals to the signals stored in the memory 756 to determine the criticality of the alarm tone. If the digital signals match (at 816) any of the signals stored in the memory 756, the microprocessor 748 transmits (at 820) a signal, which includes the criticality of the audible tone, to the central station 26 and/or illuminate the light outside of the room 18. Alternatively, the microprocessor 748 can transmit a signal, which includes the criticality of the audible tone and the data, such as room number, and/or the bed number, and/or the patient number, that was entered by the user above, to the central station 26, either directly or via the network 244.

The central station 26 receives the signal including the criticality of the audible tone and the data, such as room number, and/or the bed number, and/or the patient number, that was entered by the user above, to alert the medical personnel that the patient monitor 704 is in an alarm state and to inform the medical personnel of the severity of the alarm condition. The central station 26 displays and/or generates (at 824) a message and/or an audible tone to alert the medical personnel of the criticality of the audible tone generated by the patient monitor 704 and of the room number in which the patient monitor is located, and/or bed number that the patient monitor is assigned to, and/or patient number that the patient monitor is assigned to. The medical personnel can review the patient's data at the central station 26 prior to attending the patient and can request assistance as necessary.

In addition, the central station 26 can generate (at 828) a signal that can be transmitted to a pager 70 and/or remote display 74 to alert remote medical personnel of the critical nature of the assistance needed in room 18. The pager 70 and/or remote display 74 can display the room number, and/or the bed number, and/or the patient number with the identification of the critical nature of the audible tone (e.g., brighter, bolder, flashing message and/or louder, different tone, faster beeping, audible tone) to inform the medical personnel of the location where his or her assistance is needed. The audible tone will continue to be transmitted to the central station 26 until the alarm condition is resolved at the patient monitor 704. Alternatively, the audible tone can be silenced at the central station 26 by activation of a button or like device.

In additional embodiments, the alert device 700 can be configured to receive and transmit the data from the patient monitor 704 using other communication technologies, such as radio frequency, infra-red, ultrasonic, or any other type of wired or wireless system, whether implemented in hardware or software. The use of these communication technologies is described above with respect to an earlier described embodiment of the invention. A person having ordinary skill in the art would understand how to modify the embodiment illustrated in FIGS. 13-16 to include other communication technologies based on the description of the embodiment described in at least FIGS. 9-12.

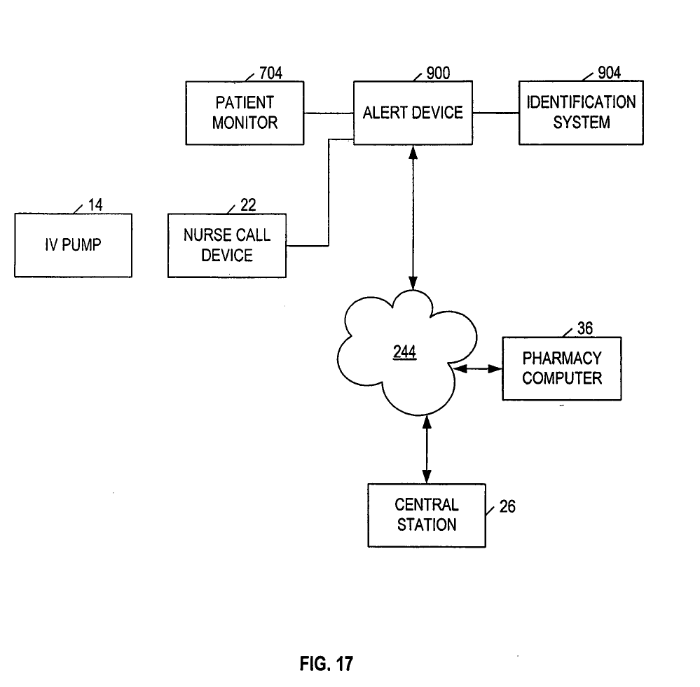

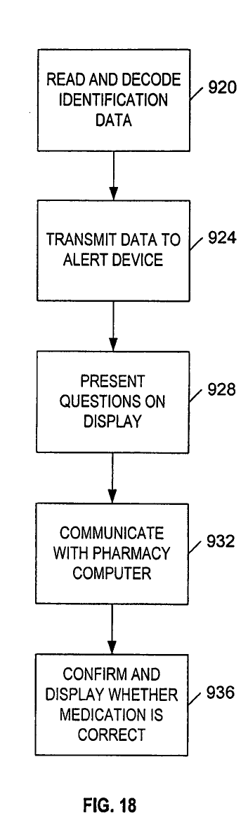

FIGS. 17-18 illustrate another embodiment of the invention as alert device 900. Other embodiments that include fewer or more terminals or components than are shown in FIG. 17 are also encompassed by the invention. FIG. 17 illustrates a network-based system that allows communication between various devices, systems, and terminals via the network 244. The network 244 can be built according to any networking technology or topology or combinations of technologies and topologies and may include multiple sub-networks. The alert device 900, the central station 26, the pharmacy computer system 36, and other computer terminals and systems (not illustrated) can be connected to the network 244. Connections between the alert device 900, the central station 26, the pharmacy computer system 36, and the other computer terminals and systems can be made through local area networks (“LANs”), wide area networks (“WANs”), public switched telephone networks (“PSTNs”), Intranets, the Internet, and other networks. In a hospital or medical care facility, communication between the systems and devices may be made through the Health Level Seven (“HL7”) protocol with any version and/or other required protocol. HL7 is a standard protocol which specifies the implementation of interfaces between two computer applications (sender and receiver) from different vendors for electronic data exchange in health care environments. HL7 allows health care institutions to exchange key sets of data from different application systems. Specifically, HL7 defines the data to be exchanged, the timing of the interchange, and the communication of errors to the application. The formats are generic in nature and must be configured to meet the needs of the two applications involved.

The alert device 900 can include fewer or more components and/or systems described above with respect to any of the described embodiments. The alert device 900 includes a timer or timing system (not shown), such as a chronometer. The alert device 900 includes an identification system 904, such as a bar code scanning system, to input information to the alert device. For example, medical personnel can order medication for the patient 10, and when it arrives at the patient's location, an identification label/tag (e.g., bar code) can be scanned by the identification system 904 to verify that the correct medication is to be administered to that particular patient 10. Medication can refer to any liquid, solid, or gaseous substance that is administered to a patient in a medical care facility.

The identification system 904 can electrically connect with the alert device 900 through a port or be included with the internal circuitry of the alert device 900. Generally, the identification system 904 includes a housing, which supports internal circuitry, such as a laser system, memory, decoder, and processor. In addition, many devices and equipment used in a medical care facility are provided with a bar code identification for tracking purposes (i.e., location, history, maintenance, serial number, model number, etc.).

The identification system 904 can be used to capture the patient identification and identification of all of the medical devices that enter the patient room 18 and interact with the patient 10. Medical personnel and/or a user can scan the identifications and/or manually enter the data at the alert device 900, such that the alert device 900 can store the data in a plurality of records or combine and store the data as a single record. The data and/or records can be stored in memory in the alert device 900 and/or the data and/or records can be transmitted to a computer terminal, database, or other system via the network 244. The unique identification (e.g., bar code) can include any data, such as the host device's serial number (or letters) that identify the type of device, model, and manufacturer. This functionality allows a medical care facility to identify and track all devices that were connected and/or interfaced with the patient 10. The alert device 900 can display the stored data and/or record on the display 716. In addition, the alert device 900 can be configured to generate an output of the data and/or records that can be transmitted to a printer. Further, the alert device 900 can be configured to transmit the data and/or records directly to a computer or other smart memory card. The data and/or records can be placed in the patient's paper and/or electronic medical record.

Referring to FIG. 18, medical personnel requests medication for the patient 10 from the pharmacy. The pharmacy inputs the request at the pharmacy computer 36 (or the medical personnel can electronically order the medication with access to the pharmacy computer 36). When the medication arrives at the patient location, medical personnel scan the medication identification label at the identification system 904. The identification system 904 reads and decodes (at 920) the identification label and transmits (at 924) the data to the alert device 900. The alert device presents (at 928) questions on the display (such as display 716 in one embodiment) to verify that the correct medication is at the intended location. The medical personnel uses the keypad (such as keypad 712 in one embodiment) to enter requested data and/or answers to the questions. The alert device 900 communicates (at 932) with the pharmacy computer 36 to verify that the medication is in the correct location (or for the correct patient). For example, the alert device transmits the inputted data, the identification, location identification (e.g., the room number in which the patient monitor is located, and/or bed number that the patient monitor is assigned to, and/or patient number that the patient monitor is assigned to that is programmed/stored in the alert device 900), and any other data to the pharmacy computer via the network 244 to perform the verification. The alert device 900 confirms and displays (at 936) whether the medication is at the correct location for the particular patient 10. The alert device 900 can store data, such as the medication, the date and time that the identification label was scanned, an identification of the medical personnel that performed the scan and verification process, etc.

The alert device can automatically interact with the pharmacy computer 36 to automatically reorder (based on physician recommendations) medications and IV solutions based on the date and time that the scan and verification process was performed. For example, the pharmacy computer 36 can store average usage data to estimate the length of time it takes to use the medication or IV solution, and by estimating the date and time the medication is administered (i.e., a predetermined amount of time after the scan and verification process is performed), the pharmacy computer 36 can automatically reorder the medication or IV solution. The medication or IV solution can be delivered to the location of the patient and administered without a waiting period.

Various features and advantages of the invention are set forth in the following claims.

1. A device to alert medical personnel comprising:
an audio sensor adapted to detect an alarm state audio signal from a medical device connected to a patient; and
an interface adapted to activate a medical personnel call device in response to the detection of the audio signal so that assistance can be provided to the patient.

2. The device as claimed in claim 1, wherein the audio sensor is a microphone.

3. The device as claimed in claim 1, wherein the interface is a relay.

4. The device as claimed in claim 1, further comprising an adjustable filter adapted to select a range of frequencies of the audio signal to be detected by the audio sensor for further processing.

5. The device as claimed in claim 4, further comprising a frequency counter adapted to count the number of audio signals within the selected range of frequencies, determine what, if any, additional supplies are needed, and communicate with a computer to reorder needed supplies.

6. The device as claimed in claim 1, further comprising a microprocessor adapted to sample and store a plurality of audio signals generated by the medical device, wherein the microprocessor is programmed to identify critical alarm audio signals.

7. The device as claimed in claim 1, further comprising a time delay adapted to select the time delay before the interface activates the call device.

8. The device as claimed in claim 1, further comprising a remotely located reset switch adapted to reset the interface via an infrared wireless signal.

9. The device as claimed in claim 1, further comprising a driver and a radio transmitter adapted to generate a unique signal and transmit the unique signal to a destination.

10. The device as claimed in claim 1, further comprising a radio transceiver adapted to transmit and receive signals.

11. A method of alerting personnel that a medical device is sounding an audible tone, the method comprising:
detecting a medical emergency through an audible tone generated by a medical device; and
activating a call device to transmit a signal to alert medical personnel.

12. The method as claimed in claim 11, further comprising counting the number of audible tones generated by the medical device and activating the call device when the number reaches a predetermined threshold.

13. The method as claimed in claim 11, further comprising selecting at least one of an automatic, manual, and dual mode of operation.

14. The method as claimed in claim 11, further comprising:
amplifying the signal;
using an identification system that includes a bar code scanner for medication verification;
transmitting alert data directly into a patient electronic medical record which can be stored in a computer database or printed to a central printer located at a central medical station.

15. The method as claimed in claim 11, wherein the signal includes an identification of a patient, and wherein the identification is displayed at the destination to inform the personnel of the medical device that is sounding the audible tone.

16. The method as claimed in claim 11, further comprising comparing the audible tone to a group of audible tones to determine the criticality of the audible tone relative to patient health.

17. A device to alert medical personnel comprising:
an audio sensor adapted to detect an audio signal generated by a patient monitor medical device;
a frequency counter adapted to count the number of audio signals detected by the audio sensor;
a microprocessor adapted to compare the audio signals to stored audio signals to identify the audio signal; and
an interface adapted to activate a call device after the frequency counter reaches a predetermined threshold, wherein the frequency counter includes an analog to digital converter.

18. A device to alert medical personnel comprising:
a microphone adapted to detect an audio signal generated by a medical device;
an adjustable filter adapted to pass the audio signal if the frequency of the audio signal is within a preselected range of frequencies and to ignore it if it is not;
a frequency counter adapted to count the number of audio signals passed by the adjustable filter;
a first relay adapted to activate a call device after the frequency counter reaches a predetemined threshold;
a microprocessor adapted to compare the audio signals to stored audio signals to identify the audio signal; and
a second relay adapted to activate the call device to transmit information related to the audio signal, wherein the information transmitted includes at least one of: patient number, room number, and bed number; and
a central station for receiving the information and displaying it to medical personnel.

19. A system for alerting personnel comprising:
a call device;
a destination in communication with the call device; and
an alert device including an audio sensor adapted to detect an audio signal generated by a medical device, a relay in communication with the call device, a display for outputting alert device information, a keypad in communication with the display for the inputting of information by medical personnel, and a processor adapted to activate the relay when an audio signal is detected by the audio sensor, wherein the call device transmits a signal to the destination to alert personnel of the audio signal generated by the medical device in response to activation of the relay.

20. The system as claimed in claim 19, further comprising a transceiver adapted to communication with the station and the alert device; and a comparator for comparing the detected alarm tones to a set of stored alarm tones.
